SEQUENCE LISTING (8 sequences)

SEQ ID NO: 1 - DNA, 24 bp, Artificial Sequence
  Cyclin-D1 sense primer
ggatgctggaggtctgcgaggaac

SEQ ID NO: 2 - DNA, 24 bp, Artificial Sequence
  Cyclin-D1 antisense primer
gagaggaagcgtgtgaggcggtag

SEQ ID NO: 3 - DNA, 20 bp, Artificial Sequence
  BAX sense primer
tttgcttcagggtttcatcc

SEQ ID NO: 4 - DNA, 20 bp, Artificial Sequence
  BAX antisense primer
cagttgaagttgccgtcaga

SEQ ID NO: 5 - DNA, 21 bp, Artificial Sequence
  BCL-2 sense primer
aatgcagtggtgcttacgctc

SEQ ID NO: 6 - DNA, 22 bp, Artificial Sequence
  BCL-2 antisense primer
ggatagcagcacaggattggat

SEQ ID NO: 7 - DNA, 19 bp, Artificial Sequence
  GAPDH housekeeping gene sense primer
gaaggtgaaggtcggagtc

SEQ ID NO: 8 - DNA, 20 bp, Artificial Sequence
  GAPDH housekeeping gene antisense primer
gaagatggtgatgggatttc

(19) United States
(12) Patent Application Publication
(10) Pub. No.: US 2014/0051754 A1
(43) Pub. Date: Feb. 20, 2014

(54) ZINC N-ACETYL TAURINATE FOR THE TREATMENT OF PROSTATE CANCER

(71) Applicant: Jean Pierre Durlach, Neuilly Sur Seine (FR)

(72) Inventor: Jean Pierre Durlach, Neuilly Sur Seine (FR)

(73) Assignee: TRI-INOV, Neuilly Sur Seine (FR)

(21) Appl. No.: 14/110,627

(22) PCT Filed: Apr. 5, 2012
(86) PCT No.: PCT/FR2012/050739

§ 371 Date: Oct. 8, 2013

(30) Foreign Application Priority Data
Apr. 8, 2011 (FR) 1153055

(51) Int. Cl. A61K 31/315

(52) U.S. Cl. CPC A61K 31/315
USPC 514/494

(57) ABSTRACT

The present invention relates to Zn N-acetyl taurinate of formula (I): [CH₃—CO—NH—CH₂—CH₂—SO₃]⁻₂Zn²⁺ for the use thereof for treating prostate cancer

The subject of the present invention is a novel use of zinc N-acetyltaurinate.

Zinc N-acetyltaurinate belongs to the family of taurine derivatives having improved neuromuscular activity, which are described in patent FR 2 384 751. The use of zinc N-acetyltaurinate for preventing and/or treating diseases with lipofuscin accumulation due to aging or to oxidative stress has been described in application WO 2009/112758.

It has now been found, surprisingly, that zinc N-acetyltaurinate (ATA-Zn) can be used as anticancer agent for the treatment of prostate cancer.

Thus, the subject of the present invention is zinc N-acetyltaurinate of formula:

[CH₃—CO—NH—CH₂—CH₂—SO₃]⁻₂Zn²⁺

for treating prostate cancer.

The subject of the invention is also the use of zinc N-acetyltaurinate for preparing a medicament that is of use for treating prostate cancer.

The zinc N-acetyltaurinate is prepared by reacting acetic anhydride with taurine in the presence of zinc acetate according to a process analogous to that described for the preparation of sodium N-acetyltaurinate by M. Terakoa (Hoppe-Seyler Zeitschrift für Physiologische Chemie, 145, 242 (1925)).

Zinc N-acetyltaurinate dihydrate is preferably used.

For the administration to patients suffering from prostate cancer, the zinc N-acetyltaurinate is advantageously mixed, as an active ingredient, with a pharmaceutically acceptable excipient commonly used for preparing pharmaceutical compositions that can be administered orally, parenterally or locally.

The anticancer activity of ATA-Zn has been demonstrated using cancer cells and normal cells of the prostate.

The tests carried out have shown that ATA-Zn has an antiproliferative effect on cancer cells of the prostate, whereas it has no effect on normal cells of the prostate.

Furthermore, it has been shown that this antiproliferative effect is concomitant with an arrest of the cell cycle via the repression of cyclin-D1 mRNA and with an increase in apoptosis via positive transcriptional regulation of the Bax/Bcl-2 apoptosis index.

Moreover, it has been shown that ATA-Zn is nontoxic at concentrations below 10⁻³M.

The invention will now be described in greater detail via the preparation and the tests hereinafter.

### PREPARATION

20.25 g of taurine and 17.5 g of dry pure zinc acetate were mixed together and 50 g of pure water were added.

The resulting suspension was heated to a temperature between 65 and 75° C. and 45 g of acetic anhydride were added to this suspension and the mixture was then heated to 100-105° C. 100 ml of anhydrous ethanol were then added to the resulting reaction mixture at a temperature between 70 and 75° C.

30±3 g of the expected product were finally obtained in the form of a white powder which is soluble in water and poorly soluble in ethanol (yield by weight: 36.25%).

Analysis (in percentages)

| Analysis | Calculated | Found |
|---|---|---|
| C | 24.16 | 22.75 |
| H | 4.05 | 4.60 |
| N | 7.04 | 5.73 |
| Zn* | 16.4 | 16.7 |

*assaying of Zn with EDTA

### In Vitro Tests

a) Materials and Methods

In the tests hereinafter, the cell cultures and the culture media hereinafter were used:

### Cell Cultures and Cell Media

The LNCaP prostate cancer cell line, deposited in the ATCC under No. CRL-1740, was used.

The cell culture medium was RPMI 1640 complete growth medium (ATCC No. 30-2001), containing 2 mM L-glutamine, 10 mM HEPES and 1.0 mM sodium pyruvate, to which the following were added:

1) sodium carbonate and glucose in amounts sufficient to obtain a sodium carbonate concentration of 20 mM and a glucose concentration of 25 mM,

2) 90% by volume of a mixture of antibiotics (10 μg/ml of gentamycin, 100 IU of penicillin, 100 μg/ml of streptomycin), and

3) 10% by volume of fetal calf serum.

Normal prostate epithelial cells (PrECs) and the PrEC growth medium sold by the company Cambrex Bioscience (Walkersville, Md.) were used by way of comparison.

### Cell Treatment

For the dose-dependence studies, the cells (50% confluent) were treated with ATA-Zn at the concentrations of 10⁻³, 10⁻⁴, 10⁻⁵, 10⁻⁶, 10⁻⁷, 10⁻⁸ and 10⁻⁹M, for 24 hours, 48 hours and 72 hours in their respective growth medium. After 48 h of treatment, the cells were harvested and counted in the pellet. In parallel, the cell lysates were prepared in order to extract the mRNA for RT-Q-PCR and Western blotting.

### Cell Count

In order to evaluate the number of cells, the cell pellets were mixed with 1 ml of their respective medium and the cell viability and the number of cells were evaluated by means of a Trypan blue exclusion test.

### RT-O-PCR (Real-Time Polymerase Chain Reaction)

The mRNA was extracted from the cells in culture using the “SV Total RNA isolation” kit from the company Promega and according to the procedure hereinafter:

The cells are harvested by drawing up the culture medium and washed with PBS (3 times 4 ml) at ambient temperature for 5 min.

175 μl of SV RNA lysis buffer are then added to the washed cells, and then the ball of RNA is dispersed and mixed thoroughly using a vortex.

The operation is repeated several times and the cell lysate obtained is placed in 1.5 ml tubes. Up to 1×10⁶ cells are readily lysed in 175 μl of the lysis buffer used.

350 μl of SV RNA dilution buffer are then added to the 175 μl of lysate. Mixing is carried out by turning the tubes over 3 to 4 times.

The tubes are then placed in a water bath at 70° C. for a maximum of 3 min.

Next, centrifugation is carried out for 10 min at 13000 g and at ambient temperature. The clear supernatant obtained is recovered in sterile 1.5 ml microtubes. 200 μl of 95% ethanol are then added and mixing is carried out by pipetting 3 to 4 times.

The alcoholic mixture obtained above is added to the separation column provided with the kit, centrifugation is carried out at 13000 g for 1 min and 600 μl of washing solution are added, and centrifugation is again carried out at 13000 g for 1 min.

The DNase solution is prepared as a mixture of 40 μl of extraction buffer, 5 μl of MnCl₂ and 5 μl of DNase.

50 μl of the DNase solution are added to the separation column membrane and left to incubate for 15 min at ambient temperature.

200 μl of SV DNase stop solution are then added, centrifugation is carried out for 1 min at 13000 g, 600 μl of washing solution are added, centrifugation is carried out once again at 13000 g for 1 min, then 250 μl of washing solution are added and centrifugation is carried out at 13000 g for 2 min. The columns are placed in the sterile elution tubes provided in the kit, 100 pl of water provided in the kit are added, and centrifugation is carried out for 1 min at 13000 g in order to elute the mRNA. The mRNA thus recovered is stored at −80° C.

The quality of the mRNA obtained is controlled and the concentration of mRNA in the sample obtained is determined according to conventional procedures well known to those skilled in the art.

A reverse transcription of the RNA was then carried out using the ready to go kit (kit known as “Ready To Go, You Prime First Strand Beads” commercially available from Amersham Biosciences, Piscataway, United States) in accordance with the supplier's instructions using 3 μg of RNA.

The “LightCycler-Fast Start DNA Master SYBR Green I” test commercially available from Roche Diagnostics, Meylan, France, was used to quantify the gene expression by means of a real-time polymerase chain reaction (PCR). The PCR (program: 95° C., 30 seconds; 40 cycles: 95° C., 5 seconds; 60° C., 35 seconds) was carried out using a “Mastercycler ep realplex” apparatus sold by the company Eppendorf, Hamburg, Germany.

The expression of the target gene was standardized relative to the GAPDH housekeeping gene. The 2^{-ΔΔCt} method was applied in order to calculate the relative gene expression.

The following primers were used for the PCR:

CYCLIN-D1:
sense primer:
5′-GGATGCTGGAGGTCTGCGAGGAAC 3′;
antisense primer:
5′-GAGAGGAAGCGTGTGAGGCGGTAG-3′;
BAX:
sense primer:
5′-TTTGCTTCAGGGTTTCATCC-3′;
antisense primer:
5′-CAGTTGAAGTTGCCGTCAGA-3′;
BCL-2:
sense primer:
5′-AATGCAGTGGTGCTTACGCTC-3′;
antisense primer:
5′-GGATAGCAGCACAGGATTGGAT-3′;
GAPDH:
sense primer:
5′-GAAGGTGAAGGTCGGAGTC-3′;
antisense primer:
5′-GAAGATGGTGATGGGATTTC-3′.

### Western Blotting

The cells were lysed in the buffer for the radioimmunoprecipitation test, having the composition hereinafter:

50 mM Tris-HCl, pH 7.4, 150 mM NaCl, Triton X-100 at 1%, SDS at 0.1%, 50 mM NaF, 2 mM Na₃VO₄, 100 nM okadaic acid, 25 mM β-glycerophosphate, 1 mM phenylmethylsulfonyl fluoride, protease inhibitor cocktail from the company Sigma containing:
2 mM AEBSF (4-(2-aminoethyl)benzenesulfonyl fluoride);
0.3 μM aprotinin;
130 μM bestatin;
1 mM EDTA;
14 μM E-64;
1 μM leupeptin.

The proteins were then separated by SDS-PAGE and were transferred onto a Hybond-P PVDF membrane (Amersham Biosciences, Pantin, France). The membranes were incubated for 1 hour at ambient temperature with a blocking solution consisting of skimmed milk powder at 5% in TBS-T buffer (50 mM Tris-HCl, pH 8.0, 150 mM NaCl, Tween-20 at 0.1%).

The membranes were then incubated with rabbit primary antibodies directed against cyclin-D1 (No. 29785); the markers Bax (No. 1063.1) and BCL-2 (No. 1017-1); Nrf-2 (No. 2178-1) and SOD-1 (No. 2018-1) all sold by the company Epitomies under the references indicated above, and the A2103 anti-actin, N-terminal, antibodies sold by the company Sigma-Aldrich (Saint Quentin Fallavier, France) in the blocking solution overnight at 4° C. The said antibodies were diluted in accordance with the manufacturer's instructions to 1/1000. The anti-actin antibodies served to standardize the membranes. The membranes were washed three times in TBS-T buffer, and were then incubated with goat anti-rabbit IgG antibodies conjugated to horseradish peroxidase (1:5000) (Santa Cruz, Calif., United States) in the blocking solution for 1 hour at ambient temperature.

After three washes, the membranes were developed using the enhanced chemiluminescence detection system known as “ECL Prime Western Blotting Detection Reagent”, sold by Amersham Biosciences (Pantin, France).

b) Test 1: Cell Counts
LNCaP Cancer Cells

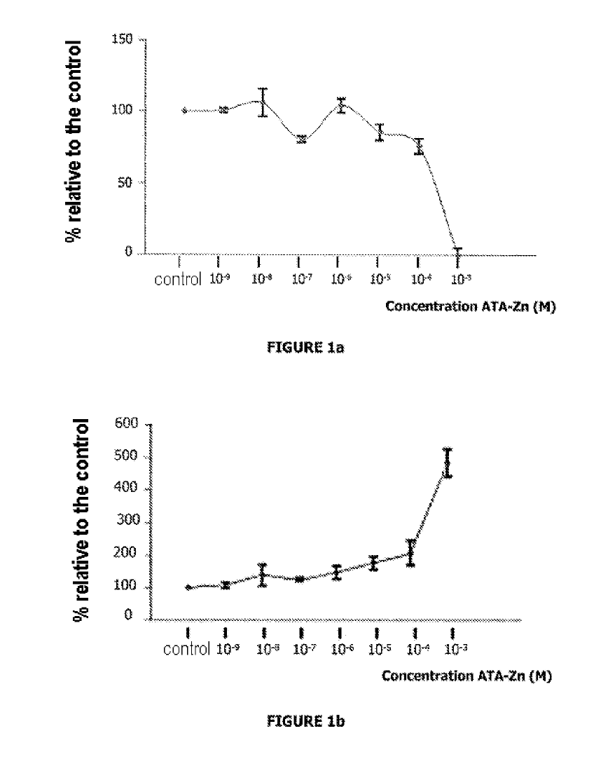

The viable cells and the non-viable cells were counted after Trypan blue exclusion. After 48 hours of incubation, it was noted that the ATA-Zn reduced the number of cells at each dose; a maximum reduction of 25% was observed at the highest concentration (10⁻⁴). The mortality evolved in the opposite direction, with a maximum increase of about 50%. The concentration of 10⁻³M was toxic. The results obtained are reported on the graphs of FIGS. 1a and b, which give the percentage of cancer cells relative to the control (cell culture without ATA-Zn) as a function of the concentration of ATA-Zn. FIG. 1a relates to the viable cells and FIG. 1b relates to the non-viable cells.
Normal PrECs

The same procedure was carried out with the normal prostate epithelia cells (PrECs) and it was noted that the ATA-Zn had no effect on the number of cells even after 72 hours of incubation, with the exception of the ATA-Zn dose of 10⁻³M which was toxic to the cells, said cells being completely dead after 72 hours of incubation with 10⁻³M ATA-Zn.

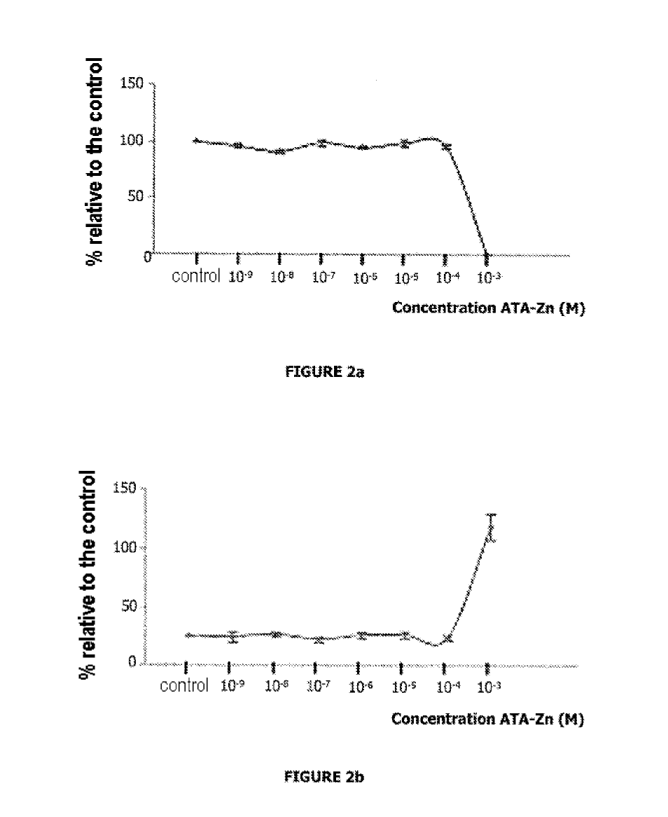

The results obtained are reported on the graphs of FIGS. 2a and 2b, which give the percentage of normal cells relative to the control (cell culture without ATA-Zn) as a function of the concentration of ATA-Zn. FIG. 2a relates to the viable cells and FIG. 2b relates to the non-viable cells.

c) Test 2: Decrease in the Cyclin-D1 mRNA

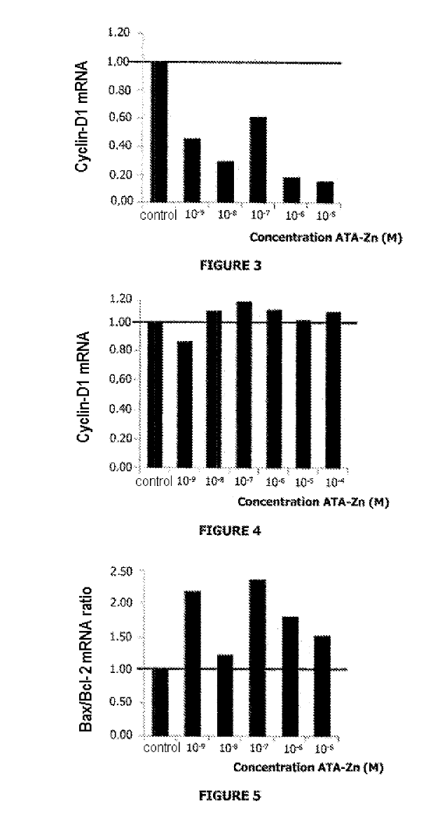

LNCaP cells were incubated with various concentrations of ATA-Zn (10⁻⁵-10⁻⁹M) for 48 hours. The amount of cyclin-D1 mRNA was measured by RT-Q-PCR according to the procedure previously described in the section entitled “Materials and methods”. It was noted that ATA-Zn reduces the synthesis of cyclin-D1 mRNA after 24 hours of exposure, as shown by the results reported on the graph of FIG. 3, which gives the amount of cyclin-D1 mRNA produced as a function of the concentration of ATA-Zn.

The same test was repeated with the normal cells and it was noted that the ATA-Zn had no effect on the synthesis of cyclin-D1 mRNA, as shown by FIG. 4.

d) Test 3: Increase in the Bax/Bcl-2 Ratio

LNCaP cells were incubated with various concentrations of ATA-Zn (10⁻⁵-10⁻⁹M) for 48 hours. The change in the synthesis of Bax mRNA and Bcl-2 mRNA was measured by RT-Q-PCR according to the procedure described previously in the section entitled “Materials and methods”. It was noted that ATA-Zn greatly increased the Bax/Bcl-2 ratio, as shown by the results reported on the graph of FIG. 5, which gives the Bax/Bcl-2 ratio as a function of the concentration of ATA-Zn.

The same test was carried out with PrECs. With these normal cells, the accumulation of Bax and of Bcl-2 was impossible to determine because the base amount was too low for good amplification by PCR. It may be concluded from this that apoptosis is very low for normal PrECs under the basal conditions and that it is not influenced by ATA-Zn.

e) Test 4: Influence of ATA-Zn on the Synthesis of Nrf-2 and SOD-1 Proteins

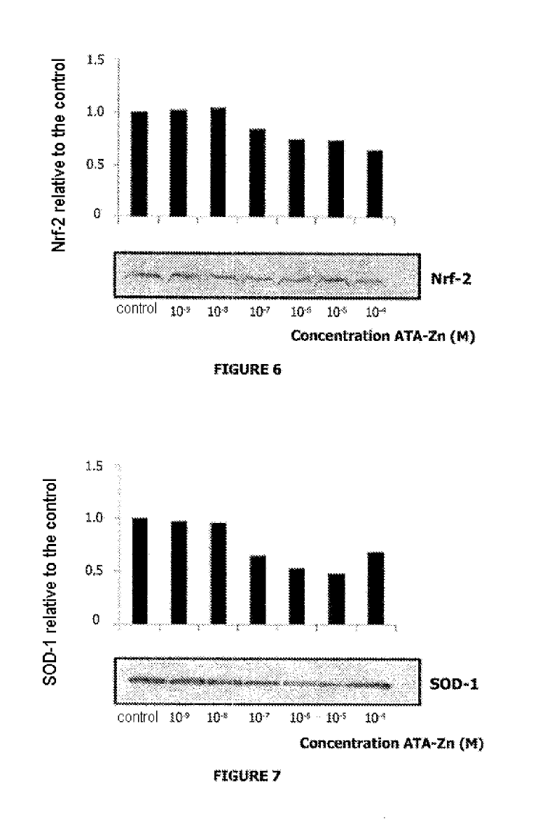

LNCaP cells were incubated with a range of ATA-Zn concentrations for 48 hours. The amount of Nrf-2 and SOD-1 proteins were then measured by Western blotting. The quantification was carried out with the “Quantity One” software and the results were standardized relative to the actin data. The results obtained are reported in FIG. 6 (Nrf-2 protein) and FIG. 7 (SOD-1 protein).

These results show that ATA-Zn decreases the level of the SOD-1 protein via an ARE (antioxidant response element)—mediated mechanism since the Nrf-2 protein is also repressed. The Nrf-2 protein is a transcription factor which is affected by the intracellular redox state. It regulates the transcription of genes which have an antioxidant response element in their promoter. Thus, it is thought that ATA-Zn modifies the oxidative status of cancer cells, causing a decrease in the growth and/or an increase in the apoptosis of the cells.

1-2. (canceled)

3. Method of treatment of prostate cancer, comprising the administration of an effective amount of zinc N-acetyltaurinate of formula:
[CH₃—CO—NH—CH₂—CH₂—SO₃]⁻₂Zn²⁺ to a patient in need thereof.

4. The method according to claim 3 wherein the patient is human.
